(19) United States
(12) Patent Application Publication
(10) Pub. No.: US 2012/0135074 A1
(43) Pub. Date: May 31, 2012

(54) High-Strength Testosterone Undecanoate Compositions

(76) Inventors: Chandrashekar Giliyar, Salt Lake City, UT (US); Basawaraj Chickmath, Salt Lake City, UT (US); Chidambaram Nachiappan, Sandy, UT (US); Mahesh V. Patel, Salt Lake City, UT (US); Srinivansan Venkateshwaran, Salt Lake City, UT (US)

(21) Appl. No.: 12/957,206

(22) Filed: Nov. 30, 2010

(51) Int. Cl. A61K 9/52; A61K 9/48; A61P 15/00; A61K 31/568

(52) U.S. Cl. 424/457; 514/178; 424/451

(57) ABSTRACT

The present disclosure is drawn to pharmaceutical compositions and oral dosage capsules containing testosterone undecanoate, as well as related methods of treatment. In one embodiment, the present invention provides for a pharmaceutical composition that includes a therapeutically effective amount of testosterone undecanoate and a solubilizer. The testosterone undecanoate is solubilized in the composition and is present in an amount such that it comprises about 14 wt % to about 35 wt % of the total composition.

### FIELD OF THE INVENTION

The present invention relates to testosterone undecanoate containing pharmaceutical compositions and oral dosage capsules as well as associated methods of treatment. Accordingly, this invention involves the fields of chemistry, pharmaceutical sciences, medicine and other health sciences.

### BACKGROUND OF THE INVENTION

Male hypogonadism is a serious condition affecting mostly aging men. The common reasons for hypogonadism in men could be physiological abnormality involving among other factors, improper functioning or growth of the gonads and/or the pituitary-hypothalamus regulatory systems, and aging. Many of the abnormalities that are identified to be commonly associated with the low or decreased testosterone levels include impaired sexual function and/or libido, metabolic syndrome which may be a combination of abdominal obesity, high blood pressure, insulin resistance, lipid disorders; high risk of cardiovascular diseases; reduced bone mass/mineral density and muscle weakness and or degeneration affecting the musculoskeletal system. Other effects of low testosterone levels include negative changes in body composition, depression and other psychological disorders. The average human male produces 4-7 mg of testosterone per day in a circadian pattern, with maximal plasma levels attained in early morning and minimal levels in the evening. It is generally recognized that in a normal adult man of age 17 to 54 years, the serum total testosterone (T) is between about 300 ng/dL (about 10.5 nmol/L) to about 1100 ng/dL (about 38.4 nmol/L) and this range is referred to as the eugonadal range. Restoration of testosterone levels to the eugonadal range typically corrects many of the cited clinical abnormalities associated with hypogonadism or low testosterone levels.

While oral administration is the most preferred and patient friendly route for administration, the effective oral delivery of testosterone as testosterone and its esters remains a challenge. This is due to extremely poor bioavailability of testosterone, which requires very high dosing as well as frequent dosing due to the short serum half-life. These problems with orally administered testosterone products are primarily due to first pass metabolism. Further, direct oral delivery of testosterone has also been known to cause enzyme induction resulting in potential drug-drug interactions.

Currently, modified testosterones, in form of a methyl analogue of testosterone, and as an undecanoate ester, testosterone undecanoate (TU) are available for oral administration for patients in need of testosterone therapy. However, liver damage including cholestasis, peliosis hepatitis, nodular regenerative hyperplasia, and primary hepatic tumors has been reported with use of methyl testosterone. Testosterone Undecanoate is a prodrug which gets converted to testosterone in vivo. Testosterone undecanoate containing products are available in some countries as liquid filled soft-gelatin capsule containing 40 mg of fully solubilized testosterone undecanoate. Testosterone undecanoate is extremely lipophilic (calculated logP of ˜6.5) with a water solubility of <0.3 ng/ml and a melting point around 62° C. It is generally believed that in order to promote lymphatic absorption for better safety profile and to facilitate effective oral delivery of testosterone undecanoate, the testosterone undecanoate must be solubilized (i.e. not present as crystals).

Accordingly, research continues into the development of testosterone oral delivery products that can have high drug load and provide for single unit oral dosage forms.

### SUMMARY OF THE INVENTION

The present disclosure is drawn to pharmaceutical compositions and oral dosage capsules containing testosterone undecanoate, as well as related methods of treatment. In one embodiment, the present invention provides for a pharmaceutical composition that includes a therapeutically effective amount of testosterone undecanoate and a solubilizer. The testosterone undecanoate is solubilized in the composition and is present in an amount such that it comprises about 14 wt % to about 35 wt % of the total composition.

In another embodiment, a pharmaceutical oral dosage capsule is provided that includes a pharmaceutical composition of the present invention disposed in a capsule shell. In yet a further embodiment, a pharmaceutical oral dosage capsule is disclosed that includes a therapeutically effective amount of testosterone undecanoate, and a solubilizer. The oral dosage capsule can include at least 50 mg of testosterone undecanoate and can be formulated to have a ratio of testosterone undecanoate to the volume of the capsule fill of about 80 mg/mL to about 750 mg/mL. Further, the oral dosage capsule can provide in vitro release of at least about 75 wt % of the testosterone undecanoate during the first 120 minutes when tested using about 1000 mL of 8% w/v Triton X-100 in water maintained at about 37±1° C. taken in a USP-Type II dissolution apparatus set at 100 rpm.

Still further embodiments of the present invention including, methods of treating a human male suffering from testosterone deficiency, are provided. The methods can include administering one or more of the pharmaceutical compositions and/or oral dosage capsules of the present disclosure.

### DETAILED DESCRIPTION

Before the present testosterone undecanoate compositions, oral dosage capsules and related methods of use are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

### DEFINITIONS

As used herein, the term “treatment,” when used in conjunction with the administration of pharmaceutical compositions and oral dosage capsules containing testosterone undecanoate, refers to the administration of the oral dosage capsules and pharmaceutically acceptable composition to subjects who are either asymptomatic or symptomatic. In other words, “treatment” can both be to reduce or eliminate symptoms associated with a condition or it can be prophylactic treatment, i.e. to prevent the occurrence of the symptoms. Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Furthermore, the term “dosage form” can include one or more formulation(s) or composition(s) provided in a format for administration to a subject. When any of the above terms is modified by the term “oral” such terms refer to compositions, formulations, or dosage forms formulated and intended for oral administration to subjects.

As used herein, the term “fatty acid” refers to unionized carboxylic acids with a long unbranched aliphatic tail (chain), either saturated or unsaturated, conjugated or non-conjugated.

Unless otherwise specified, the term C₈ to C₂₂ fatty acid glycerides refers to a mixture of mono-, di-, and/or tri-glycerol esters of medium to long chain (C₈ to C₂₂) fatty acids.

As used herein, the term “dispersant” refers to any pharmaceutically acceptable additive that enables the contents of the compositions and/or oral dosage capsules to finely disperse in an aqueous medium. The extent of dispersion in an aqueous medium can be determined spectrophotometrically from the absorbance exhibited by the dispersion at a wavelength of about 400 nm. For example, the dispersion of the composition (with or without the testosterone undecanoate) of the current invention in about 0.2 mM sodium lauryl sulphate solution in water, has an absorbance of about 0.6 or less at about 400 nm wavelength, when the ratio of the composition to the sodium lauryl sulphate solution is about 1:2000. In a specific embodiment, the dispersion of the composition (with or without the testosterone undecanoate) of the current invention in about 0.2 mM sodium lauryl sulphate solution in water, has an absorbance of about 0.3 or less at about 400 nm wavelength, when the ratio of the composition to the sodium lauryl sulphate solution is about 1:5000. In another embodiment, the composition can produce a fine dispersion upon dilution in an aqueous medium without the need of a hydrophilic surfactant.

Further, as used herein, the dispersant of the current invention is at least one selected from the group of hydrophilic surfactant or lipophilic surfactant. In one embodiment, the dispersant includes a hydrophilic surfactant.

As used herein, the term “solidifying agent” or “solidifying additive” are used interchangeably and refer to a pharmaceutically acceptable additive that is in a solid physical state at 20° C. Similarly, a “solid lipophilic additive” refers to a lipophilic compound that is in a solid physical state at 20° C. and/or renders the composition or dosage form solid or semi-solid.

As used herein, the terms “solubilized” and “solubility,” when used to describe the state of testosterone undecanoate with respect to a composition or oral dosage form, refers to the absence of testosterone undecanoate crystals in the composition or oral dosage form when observed under hot-stage microscope over a temperature of about 25° C. to about 65° C., or the absence of crystalline testosterone undecanoate melting related peak (about 62 to about 65° C.). when the composition or oral dosage form is subjected to differential scanning calorimetry. Similarly, the solubility of testosterone undecanoate in a particular compound, e.g. a solubilizer, is the amount of testosterone undecanoate dissolved to form a visibly clear solution at a specified temperature such as about 25° C. or about 37° C.

As used herein, the term “lipophilic,” refers to compounds that are not freely soluble in water; and the term “lipophilic surfactant” refers to surfactants that have HLB values of 10 or less. Conversely, the term “hydrophilic” refers to compounds that are soluble in water; and term “hydrophilic surfactant” refers to surfactants that have HLB values of more than 10.

As used herein, “subject” refers to a mammal that may benefit from the administration of a drug composition or method of this invention. Examples of subjects include humans. In one aspect, the subject can be a human male.

The term “oral administration” represents any method of administration in which an active agent can be administered by swallowing, chewing, or sucking of the dosage form. The composition of the current inventions can be admixed with food or drink prior to being orally consumed.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the term “delayed release” refers to the release into an aqueous solution of the testosterone undecanoate from the composition or oral dosage form in a time delayed manner attributed either to the inherent nature of the composition or to a coating which may surround the composition or the oral dosage form. A traditional gelatin or non-gelatin non-enteric capsule shell does not alone constitute a delayed release. In one embodiment, the delayed release is such that about 20% or less of the testosterone undecanoate is released within the first 15 minutes after the composition is contacted by the aqueous solution.

As used herein, the testosterone deficiency or hypogonadism in a male human subject refers to a condition wherein the average baseline plasma testosterone concentration (T-C_{avg-B}) is about 300 ng/dL or less. However in some instances, testosterone deficiency or hypogonadism in a male human subject refers to a condition wherein the average baseline plasma testosterone concentration is about 400 ng/dL or less. The terms “plasma testosterone concentration,” “testosterone concentration in the blood,” and “serum testosterone concentration” are used interchangeably and refer to the “total” testosterone concentration which is the sum of the bioavailable testosterone including free and protein-bound testosterone concentrations.

As used herein, of the average plasma testosterone concentration can be determined using methods and practices known in the art. For example, the average baseline plasma testosterone concentration of a human male is the arithmetic mean of the total plasma testosterone concentrations determined on at least two consecutive time points that are reasonably spaced from each other, for example from about 1 hour to about 168 hours apart. In a particular case, the plasma testosterone concentration can be determined on at least two consecutive times that are about 12 hours to about 48 hours apart. In another particular method, the plasma testosterone concentration of the human male can be determined at a time between about 5 o'clock and about 11 o'clock in the morning. Further, the plasma testosterone concentration can be the determined by standard analytical procedures and methods available in the art, such as for example, automated or manual immunoassay methods, liquid chromatography or liquid chromatography-tandem mass spectrometry (LC-MSMS) etc.

As used herein, the term AUC₀₋ₜ is the area under the curve of a plasma-versus-time graph determined for the analyte from the time 0 to time “t”.

As used herein, the term “C_{avg} or “C-average” is used interchangeably, and is determined as the AUC₀₋ₜ or the mean AUC divided by the time period (t). For example, C_{avg-8h} is the average plasma concentration over a period of 8 hours post-dosing determined by dividing the AUC₀₋₈ value by 8. Similarly, C_{avg-12h} is the average plasma concentration over a period of 12 hours post-dosing determined by dividing the AUC₀₋₁₂ value by 12; C_{avg-24h} is the average plasma concentration over a period of 24 hours post-dosing determined by dividing the AUC₀₋₂₄ₕ value by 24, and so on.

As used herein, the term “about” is used to provide flexibility to a numerical range endpoint by providing that a given value may be “a little above” or “a little below” the endpoint. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### INVENTION

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

In one embodiment, a pharmaceutical composition is provided that includes a therapeutically effective amount of testosterone undecanoate and a solubilizer. The testosterone undecanoate is solubilized in the composition and is present in an amount such that it comprises about 14 wt % to about 35 wt % of the total composition.

The pharmaceutical compositions of the present invention can be administered to subjects in various oral dosage capsules. In one embodiment, a pharmaceutical oral dosage capsule is provided that includes a pharmaceutical composition of the present invention disposed in a capsule shell. In yet another embodiment, a pharmaceutical oral dosage capsule can include a therapeutically effective amount of testosterone undecanoate, and a solubilizer. The oral dosage capsule can include at least 50 mg of testosterone undecanoate and can be formulated to have a ratio of testosterone undecanoate to the fill volume of the composition or capsule fill of about 80 mg/mL to about 750 mg/mL. Further, the oral dosage capsule can provide in vitro release of at least about 75 wt % of the testosterone undecanoate during the first 120 minutes when tested using about 1000 mL of 8% w/v Triton X-100 in water maintained at about 37±1° C. taken in a USP-Type II dissolution apparatus set at 100 rpm.

The compositions and oral dosage capsules of the present invention can be used to treat subjects, particularly human males, who suffer from testosterone deficiency. Accordingly, in one embodiment of the present invention, methods of treating a human male suffering from testosterone deficiency are provided. The methods can include administering one or more of the pharmaceutical compositions and/or oral dosage capsules of the present disclosure to the subject in need thereof. The administration of the pharmaceutical compositions and/or oral dosage capsules can be accomplished at various dosing intervals. In one embodiment, the administration can be done once-a-day, or once every 24 hours. In another embodiment, the administration can be done twice-a-day or once every 12 hours. In yet another embodiment, the administration can be done three times a day, or once every 8 hours. One advantage of the oral dosage capsules of the present invention is their ability to provide therapeutic effect through single dosage form administration, i.e. no need to administer multiple dosage forms in order to achieve therapeutic result.

Testosterone deficiency is typically associated with a particular condition that is the source or causes the deficiency. The compositions and oral dosage forms of the present invention can be used to treat any condition associated with testosterone deficiency, including complete absence, of endogenous testosterone. Examples of conditions associated with testosterone deficiency that can be treated using the oral dosage capsules and/or compositions of the present invention include, but are not limited to congenital or acquired primary hypogonadism, hypogonadotropic hypogonadism, cryptorchidism, bilateral torsion, orchitis, vanishing testis syndrome, orchidectomy, Klinefelter's syndrome, post castration, eunuchoidism, hypopituitarism, endocrine impotence, infertility due to spermatogenic disorders, impotence, male sexual dysfunction (MSD) including conditions such as premature ejaculation; erectile dysfunction, decreased libido, and the like, micropenis and constitutional delay, penile enlargement, appetite stimulation, testosterone deficiency associated with chemotherapy, testosterone deficiency associated with toxic damage from alcohol, testosterone deficiency associated with toxic damage from heavy metal, osteoporosis associated with androgen deficiency, and combinations thereof.

Other conditions that can be treated by the compositions and oral dosage forms disclosed herein include idiopathic gonadotropin, LHRH deficiency, or pituitary hypothalamic injury from tumors, trauma, or radiation. Typically, these subjects have low serum testosterone levels but have gonadotropins in the normal or low range. In one embodiment, the compositions or oral dosage forms may be used to stimulate puberty in carefully selected males with clearly delayed puberty not secondary to pathological disorder. In another embodiment, the compositions and oral dosage forms may be used in female-to-male transsexuals in order to maintain or restore male physical and sexual characteristics including body muscle mass, muscle tone, bone density, body mass index (BMI), enhanced energy, motivation and endurance, restoring psychosexual activity etc. In some embodiments, the testosterone undecanoate compositions and oral dosage capsules may be useful in providing hormonal male contraception.

Additionally, testosterone therapy can also be used to improve the quality of life of subjects suffering from conditions such as decreased libido, diminishing memory, anemia due to marrow failure, renal failure, chronic respiratory or cardiac failure, steroid-dependent autoimmune disease, muscle wasting associated with various diseases such as AIDS, preventing attacks of hereditary angioedema or urticaria; andropause, and palliating terminal breast cancer. In some situations, certain biomarkers such as for example, increased SHBG levels, can be used to diagnose a subject who may be in need of testosterone therapy. These biomarkers can be associated with conditions/disease states such as anorexia nervosa, hyperthyroidism, hypogonadism, androgen insensitivity/deficiency, alcoholic hepatic cirrhosis, primary biliary cirrhosis, and the like.

Subjects that can be treated by the testosterone undecanoate compositions and oral dosage capsule of the present disclosure can be any human male in need thereof. In particular, in one embodiment, the human male may be at least 14 years of age. In another embodiment, the human male is an adult of at least age 30. In a further embodiment, the subject can be an adult male of at least age 50. In yet a further embodiment, the subject can be an adult male of at least age 60.

As discussed above, the compositions and oral dosage capsules disclosed herein can be used to treat testosterone deficiency in human males. In one embodiment, the human male being treated can have an average baseline plasma testosterone concentration of about 400 ng/dL or less. In another embodiment, the human male being treated can have an average baseline plasma testosterone concentration of about 350 ng/dL or less. In another embodiment, the human male being treated can have an average baseline plasma testosterone concentration of about 300 ng/dL or less. In another embodiment, the human male being treated can have an average baseline plasma testosterone concentration of about 250 ng/dL or less. In still another embodiment, the human male being treated can have an average baseline plasma testosterone concentration of about of about 190 ng/dL or less. In still a further embodiment, the human male has an average baseline plasma testosterone concentration of about 400 ng/dL or less, along with a co-morbid condition of insulin resistance.

Further, there are several biomarkers that can be used to identify patients who need testosterone therapy through the administration of the compositions and/or dosage forms of the current invention. Accordingly, in one embodiment, the human male being treated can have a low density lipoproteins (LDL) level in greater than about 130 mg/dL of blood. In another embodiment, the human male being treated can have a high density lipoproteins (HDL) level less than about 40 mg/dL of blood. In still another embodiment, the human male being treated can have a total cholesterol level greater than about 220 mg/dL of blood. In yet a further embodiment, the human male being treated can have an average TG (triglycerides) levels greater than 250 mg/dL of blood. In one embodiment, the testosterone undecanoate dosage forms of the current invention can be administered to human male whose bioavailable or free or un-bound plasma estradiol levels are about 20 pg/mL or less. In another embodiment, dosage forms of the current invention can be administered to human male who has a ratio of the bioavailable or free or unbound plasma testosterone level to the bioavailable or free or un-bound plasma estradiol level at about 100 or less.

The testosterone undecanoate compositions and oral dosage capsules of the current invention can be administered orally to a human male who has an average body mass index (BMI) of about 28 kg/m² or more. In another embodiment, the human male has an average BMI of about 30 kg/m² or more. In another embodiment, the human male has an average BMI of about 37 kg/m² or more. In a further embodiment, the subject male being treated can have a serum sex hormone binding globulin (SHBG) levels of about 40 nmol/L or more. In yet another embodiment, the human male being treated can have a serum SHBG levels of about 60 nmol/L or more.

It was found that the pharmaceutical compositions and oral dosage capsules of the present invention have the ability to provide for increased stability of the testosterone undecanoate present in the formulation. In particular, the pharmaceutical compositions and oral dosage capsules of the present invention can provide for superior stability with respect to the degradation of the testosterone undecanoate that can occur during storage as compared to other formulation containing lower testosterone undecanoate concentration. In one embodiment, the pharmaceutical compositions and oral dosage capsules of the present invention can have increased stability such that, when stored for a period of at least three months there is at least 20% less degradation of the testosterone undecanoate as compared to testosterone undecanoate containing compositions having less than 14 wt % testosterone undecanoate. In another embodiment, the pharmaceutical compositions and oral dosage capsules of the present invention can have increased stability such that, when stored for a period of at least three months there is at least 20% less degradation of the testosterone undecanoate as compared to testosterone undecanoate containing compositions having less than 16 wt % testosterone undecanoate.

Further, it has been discovered that the pharmaceutical compositions and oral dosage capsules disclosed herein can provide therapeutically effective treatment without the need to include oils, triglycerides, and/or hydrophilic surfactants. Accordingly, in one embodiment, the pharmaceutical compositions and oral dosage capsules can be free of oil. In another embodiment, the pharmaceutical composition and oral dosage capsules can be free of triglycerides. In yet a further embodiment, the pharmaceutical compositions and oral dosage capsules can be free of hydrophilic surfactants. In yet a further embodiment, the composition can include a hydrophilic surfactant as a dispersant and the hydrophilic surfactant can be present in an amount such that it does not appreciably solubilize the testosterone undecanoate in the composition. A hydrophilic surfactant is said to “not appreciably solubilize” testosterone undecanoate when it solubilizes 5 wt % or less of the testosterone undecanoate in the composition or the dosage form. In one embodiment, a hydrophilic surfactant is deemed to “not appreciably solubilize” testosterone undecanoate when it solubilizes 2 wt % or less of the testosterone undecanoate in the composition or oral dosage capsule. In all of these embodiments, the pharmaceutical compositions and oral dosage capsules can still be capable of providing the necessary dispersion and pharmacokinetics parameters to effectively treat testosterone deficiency.

The testosterone undecanoate can be present in the pharmaceutical compositions and oral dosage capsules in amounts sufficient to comprise 14 wt % to about 35 wt % of the composition or oral dosage capsule. In one embodiment, the testosterone undecanoate can make up about 15 wt % to about 30 wt % of the composition or oral dosage capsule. In yet a further embodiment, the oral dosage capsule can comprise about 18 wt % to about 25 wt % of the composition or oral dosage capsule. The oral dosage capsules of the present application can include dosages of testosterone undecanoate of at least 50 mg. The oral dosage capsules of the present application can include dosages of testosterone undecanoate of about 80 mg to about 400 mg. In another embodiment, the oral dosage capsule can include about 120 mg to about 300 mg testosterone undecanoate. In yet a further embodiment, the oral dosage capsule can include about 150 mg to about 250 mg of testosterone undecanoate.

The solubilizers used in the pharmaceutical compositions and oral dosage capsules of the present invention play role in the ability of the formulation to provide the desired therapeutic characteristics. Solubilizers that can be used can be selected from a variety of compounds and mixtures of compounds that have the ability to facilitate loading of testosterone undecanoate. The solubilizer can comprise about 50 wt % to about 86 wt % of the composition. In one embodiment, the solubilizer can comprise about 55 wt % to about 82 wt % of the pharmaceutical composition or oral dosage capsule. In another embodiment, the solubilizer can comprise about 60 wt % to about 80 wt % of the pharmaceutical composition or oral dosage capsule. In one embodiment, the solubilizer can be such that the testosterone undecanoate can have solubility in the solubilizer, at about 37° C., of about 250 mg/g to about 750 mg/g (mg testosterone undecanoate/gram of solubilizer and testosterone undecanoate).

Non-limiting examples of solubilizers that can be used include C₈ to C₂₂ fatty acid glycerides, alcohols, omega fatty acids, and mixtures thereof. In one embodiment, the C₈ to C₂₂ fatty acid glycerides can include C₈ to C₂₂ medium and/or long chain monoglycerides, medium and/or long chain diglycerides, or mixtures of a mixture of medium and/or long chain monoglycerides and medium and/or long chain diglycerides. In another embodiment, the solubilizer can consist essentially of medium and/or long chain monoglycerides and/or diglycerides. Medium to long chain monoglycerides and diglycerides refers to compounds having chain lengths of C₈ to C₂₂. In one embodiment, the mixture of monoglycerides and diglycerides can have chain lengths of C₈ to about C₁₃. In another embodiment, the mixture of monoglycerides and diglycerides can have chain lengths of about C₁₄ to about C₂₂. When the solubilizer includes C₈ to C₂₂ fatty acid glycerides, monoglycerides can comprise at least about 40 wt % of the C₈ to C₂₂ fatty acid glycerides. In another embodiment, the monoglycerides can comprise at least about 60 wt % of the C₈ to C₂₂ fatty acid glycerides. In yet a further embodiment, the monoglycerides can comprise at least about 80 wt % of the C₈ to C₂₂ fatty acid glycerides.

Non-limiting examples of C₈ to C₂₂ fatty acid glycerides that can be used as solubilizers in pharmaceutical compositions and oral dosage capsules of the present invention include monoglycerides and/or diglycerides derived from sources such as maize oil, poppy seed oil, safflower oil, sunflower oil, coconut oil, palm kernel oil, castor oil, and mixtures thereof. Although not essential, the solubilizer can also include a triglyceride. The triglyceride can be a medium and/or long chain triglyceride, or mixture thereof, and can be present alone or with other solubilizers. The triglycerides can be selected from a variety of well known pharmaceutically acceptable triglycerides including, but not limited to vegetable oils such as peanut oil, safflower oil, sunflower oil, olive oil, castor oil, corn oil, maize oil, flax seed oil, wheat-germ oil and the like, or their hydrogenated derivatives and their mixtures thereof. Additional triglyceride sources can include animal derived oils such as fish oil, seal oil, whale oil, and the like, triglycerides of C₈-C₂₂ fatty acids or their mixtures; triglycerides of C₈-C₁₃ fatty acids; triglycerides of C₁₄-C₂₂ fatty acids. In one embodiment, the composition can include a fatty acid triglyceride and the testosterone undecanoate can comprise at least about 25 wt % of the composition. In another embodiment, the triglyceride can be castor oil. In yet a further embodiment, the castor oil can comprise about 45 wt % or less of the total composition. In yet another embodiment, the castor oil can comprise about 40 wt % or less of the solubilizer. In a further embodiment, the composition can be free of castor oil. In one embodiment of the invention, the solubilizer can include a glyceryl palmitostearate, a glyceryl stearate, a glyceryl distearate, glyceryl monostearate, or a combination there of.

In another aspect of the invention, the solubilizer can include a C₈ to C₂₂ fatty acid glycerides that is monoglycerides and/or diglycerides of capric acid, caprylic acid, or mixtures thereof. In another embodiment, the solubilizer can include a C₈ to C₂₂ fatty acid glycerides that is a monoglycerides and/or diglycerides of linoleic acid, oleic acid, or mixtures thereof. Other examples of C₈ to C₂₂ fatty acids that can be used include capric acid, pelargonic acid, caprylic acid, undecanoic acid, lauric acid, myristic acid, palmitic acid, stearic acid, oleic acid, linoleic acid, linolenic acid, arachodonic acid, eicosapentaenoic acid, docosahexanoic acid, and mixtures thereof. In one embodiment, the C₈ to C₂₂ fatty acid can be capric acid, caprylic acid, undecanoic acid, lauric acid, myristic acid, palmitic acid, stearic acid, oleic acid, linoleic acid, linolenic acid or mixtures thereof. In another embodiment, the C₈ to C₂₂ fatty acid can be selected from the group consisting of capric acid, caprylic acid, oleic acid, linoleic acid, and mixtures thereof.

In a further embodiment, the solubilizer can include an alcohol. Non-limiting examples of alcohols that can be used as solubilizers include tocopherol, ethyl alcohol, isopropanol, butanol, benzyl alcohol, ethylene glycol, propylene glycol, butanediol, glycerol, pentaerythritol, transcutol, dimethyl isosorbide, polyethylene glycol and mixtures thereof. In one embodiment, the solubilizer can be ethyl alcohol, benzyl alcohol, tocopherol, and mixtures thereof.

The pharmaceutical compositions and oral dosage capsules can also include a dispersant. In one aspect of the invention, the dispersant can be a hydrophilic surfactant having an HLB value of greater than 10, a lipophilic surfactant having an HLB value of 10 or less, or combinations thereof. In one embodiment, the compositions and oral dosage forms can include at least one hydrophilic surfactant.

When present, the hydrophilic surfactant can, but does not have to have appreciable solubilizing effect for the testosterone undecanoate present in the composition. Non-limiting examples of hydrophilic surfactants that can be included are non-ionic hydrophilic surfactants such as polysorbates, polyoxyethylene hydrogenated vegetable oils, polyoxyethylene vegetable oils; polyoxyethylene sorbitan fatty acid esters; polyoxyethylene-polyoxypropylene block copolymers; polyglycerol fatty acid esters; polyoxyethylene glycerides; polyoxyethylene sterols, derivatives and analogues thereof; reaction mixtures of polyols and at least one member of the group consisting of fatty acids, glycerides, vegetable oils, hydrogenated vegetable oils, fractionated oils and sterols; tocopheryl polyethylene glycol succinates; sugar esters; sugar ethers; sucroglycerides; mixtures thereof; alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers such as poloxamer-108, 188, 217, 238, 288, 338, 407, 124, 182, 183, 212, 331, or 335, or combinations thereof; ionic hydrophilic surfactants such as sodium dodecyl sulphate, docusate sodium; bile acid, cholic acid, deoxycholic acid, chenodeoxycholic acid, salts thereof, and mixtures thereof. In one embodiment, the pharmaceutical composition or oral dosage form can be substantially free of hydrophilic surfactants.

In one embodiment, the hydrophilic surfactant can have at least one characteristic of 1) being present in an amount such that it does not appreciably solubilize testosterone undecanoate present in the composition; or 2) the solubility of testosterone undecanoate in the hydrophilic surfactant at about 25° C., is less than 100 mg/gram or less, based on the total weight of the testosterone undecanoate and the solubilizer.

In one embodiment, the hydrophilic surfactant can have at least one characteristic of 1) being present in an amount such that it solubilizes less than 5 wt % of the testosterone undecanoate present in the composition; or 2) the solubility of testosterone undecanoate in the hydrophilic surfactant at about 25° C., is less than 100 mg/gram or less, based on the total weight of the testosterone undecanoate and the surfactant. In another embodiment, the hydrophilic surfactant can have at least one characteristic of: 1) the hydrophilic surfactant is present in an amount such that it solubilizes less than 5 wt % of the testosterone undecanoate present in the composition; or 2) the solubility of testosterone undecanoate in the hydrophilic surfactant at about 25° C., is about 50 mg/gram or less, based on the total weight of the testosterone undecanoate and the surfactant. In yet a further embodiment, the hydrophilic surfactant can have a least one characteristic of: 1) the hydrophilic surfactant is present in an amount such that it solubilizes less than 5 wt % of the testosterone undecanoate present in the composition; or 2) the solubility of testosterone undecanoate in the hydrophilic surfactant at about 25° C. about 10 mg/gram or less, based on the total weight of the testosterone undecanoate and the surfactant. In yet a further embodiment, the hydrophilic surfactant can have the characteristic of: 1) the hydrophilic surfactant is present in an amount such that it solubilizes less than 5 wt % of the testosterone undecanoate present in the composition; and 2) the solubility of testosterone undecanoate in the hydrophilic surfactant at about 25° C., is about 50 mg/gram or less, based on the total weight of the testosterone undecanoate and the surfactant.

As discussed above, in some embodiments the compositions and oral dosage capsules can include at least one lipophilic surfactant. Various lipophilic surfactants can be used including, but not limited to reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil®M 1966 CS), PEG-6 apricot kernel oil (Labrafil®M 1944 CS), PEG-6 olive oil (Labrafil®M 1980 CS), PEG-6 peanut oil (Labrafil®M 1969 CS), PEG-6 hydrogenated palm kernel oil (Labrafil®. M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol® M40), PEG-20 almond glycerides (Crovol® A40), lipophilic polyoxyethylene-polyoxypropylene block co-polymers (Pluronic® L92, L101, L121 etc.); propylene glycol fatty acid esters, such as propylene glycol monolaurate (Lauroglycol FCC), propylene glycol ricinoleate (Propymuls), propylene glycol monooleate (Myverol P-06), propylene glycol dicaprylate/dicaprate (Captex® 200), and propylene glycol dioctanoate (Captex® 800), propylene glycol mono-caprylate (Capryol® 90); propylene glycol oleate (Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (Arlacel® 186); sterol and sterol derivatives such as cholesterol, sitosterol, phytosterol, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (Arlacel 20), sorbitan monopalmitate (Span-40), sorbitan monooleate (Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; and mixtures thereof. It is important to note that some lipophilic surfactants may also function as the solubilizer component of the compositions and oral dosage forms.

In one embodiment, the lipophilic surfactant can be selected from the group consisting of propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (Arlacel 20), sorbitan monopalmitate, sorbitan monooleate, sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof.

In another aspect of the invention, the pharmaceutical compositions and/or oral dosage capsules can include a solidifying agent. As defined above, a solidifying agent is a pharmaceutically acceptable additive that is in a solid physical state at 20° C. Typically solidifying agents facilitate the solidification of the pharmaceutical compositions of the present invention at temperatures around room temperature. When present, the solidifying agent can comprise from about 0.1 wt % to about 25 wt % of the pharmaceutical composition or oral dosage capsule. In another embodiment, the solidifying agent can comprise about 2 wt % to about 20 wt % of the composition or oral dosage capsule. In yet a further embodiment, the solidifying agent can comprise about 6 wt % to about 15 wt % of the composition or oral dosage capsule. In one embodiment, the solidifying agent can melt at a temperature of about 45° C. to about 75° C. Non-limiting examples of solidifying agents that can be used include polyethylene glycols; sorbitol; gelatin; stearic acid; cetyl alcohol; cetosterayl alcohol; paraffin wax; polyvinyl alcohol; glyceryl stearates; glyceryl distearate; glyceryl monostearate; glyceryl palmitostearate; glyceryl behenate; waxes; hydrogenated castor oil; hydrogenated vegetable oil; bees wax, microcrystalline wax; sterols; phytosterols; cholesterol and mixtures thereof. In one embodiment, the solidifying agent includes a polyethylene glycol (PEG) having molecular weight from about 1000 to about 20,000 and their mixtures. In another embodiment the solidifying agent includes one or more selected from the group consisting of polyethylene glycol; gelatin; stearic acid; polyvinyl alcohol; glyceryl stearates; glyceryl distearate; glyceryl monostearate; glyceryl palmitostearate; hydrogenated castor oil; hydrogenated vegetable oil and cholesterol. In one embodiment, the pharmaceutical composition can be a solid at about 20° C.

The oral compositions of the present invention can be formulated to take any dosage form commonly known in the pharmaceutical arts such as granules, tablet or capsule. In one embodiment, the oral dosage form can be a capsule having a pharmaceutical composition of the present invention disposed therein. Both soft and hard gelatin and non-gelatin capsules can be used. The capsule size can be any size known in the art and can vary depending on the desired dosage amount. The oral dosage capsules can be immediate release, extended release, targeted release, enteric release, delayed release dosage form or combinations thereof. In a specific embodiment, the oral dosage capsule can be a delayed release dosage form. In one embodiment, the capsule can have a ratio of the amount of testosterone undecanoate to the volume of the capsule fill can be about 80 mg/mL to about 750 g/mL. In another embodiment, the capsule can have a ratio of the amount of testosterone undecanoate to the volume of the capsule fill can be about 160 mg/mL to about 375 mg/mL.

The oral dosage capsules of the present invention can be formulated such that they have distinctive release profiles. In one embodiment, an oral dosage capsule can provide in vitro release of at least about 75 wt % of the testosterone undecanoate during the first 120 minutes when tested using about 1000 mL of 8% w/v Triton X-100 in water maintained at about 37±1° C. taken in a USP-Type II dissolution apparatus set at 100 rpm. In another embodiment, the oral dosage capsule can have an in vitro release profile such that 85 wt % or less of the testosterone undecanoate is released in the first 30 minutes, when measured using about 1000 mL of 8% w/v Triton X-100 in water maintained at about 37±1° C. taken in a USP-Type II dissolution apparatus set at 100 rpm. In further embodiment, the oral dosage capsule can have an in vitro release profile such that 70 wt % or less of the testosterone undecanoate is released in the first 30 minutes, when measured using about 1000 mL of 8% w/v Triton X-100 in water maintained at about 37±1° C. taken in a USP-Type II dissolution apparatus set at 100 rpm. In an additional embodiment, the oral dosage capsule can have a in vitro release profile such that at least 35 wt % of the testosterone undecanoate is released in the first 30 minutes, when measured using about 1000 mL of 8% w/v Triton X-100 in water maintained at about 37±1° C. taken in a USP-Type II dissolution apparatus set at 100 rpm. In still an additional embodiment, the oral dosage capsule can have an in vitro release profile such that at least 40 wt % of the testosterone undecanoate is released in the first 30 minutes, when measured using about 1000 mL of 8% w/v Triton X-100 in water maintained at about 37±1° C. taken in a USP-Type II dissolution apparatus set at 100 rpm.

In one aspect, the dosage form can comprise two or more of populations of testosterone undecanoate compositions of the present invention. In one embodiment, at least one of the populations can be formulated to start releasing testosterone undecanoate immediately into a surrounding aqueous medium. In another embodiment, at least one the populations can be formulated to start releasing testosterone undecanoate after at least 2 hours. In another embodiment, at least one the populations can be formulated to release testosterone undecanoate after about 4 hours, or after about 6 hours, or after about 8 hours, or after about 10 hours.

In yet a further embodiment, at least one of the populations can be formulated to start releasing testosterone undecanoate immediately after oral administration to a human. In one particular case, at least one of the populations can be formulated to start releasing testosterone undecanoate in the duodenal region after oral administration to a human. In another particular case, at least one of the populations can be formulated to start releasing testosterone undecanoate in the small intestine after oral administration to a human.

In yet a further embodiment, at least one of the populations includes a pH sensitive substance. In a particular case, at least one of the populations can be formulated to start releasing testosterone undecanoate at a pH of from about 1.0 to about 3.4. In another particular case, at least one of the populations can be formulated to start releasing testosterone undecanoate at a pH of from about 3.5 to about 5.5. In another particular case, at least one of the populations can be formulated to start releasing testosterone undecanoate at a pH of from about 5.6 to about 6.8. In another particular case, at least one of the populations can be formulated to start releasing testosterone undecanoate at a pH about 7.0 or more.

In yet another aspect, the dosage form comprising two or more of populations of testosterone undecanoate compositions of the present invention is a capsule. In a particular case, the dosage form is a capsule in capsule dosage form. In another particular case the dosage form is a tablet in capsule dosage form. In another particular case, the dosage form is a granules or pellets or tablets or minitablets disposed in a capsule.

The oral dosage capsules of the present invention can be formulated such that, when administered to a human male they provide a serum total testosterone C_{avg} ranging about 300 ng/dL to about 1100 ng/dL. In another embodiment, the oral dosage capsule can be formulated such that, upon single administration to a human male, they provide a serum total testosterone C_{avg} ranging about 350 ng/dL to about 800 ng/dL. In another embodiment, the oral dosage capsule can be formulated such that, upon single administration to a human male, they provide a serum total testosterone C_{avg} ranging from about 400 ng/dL to about 600 ng/dL. It is noted that such C_{avg} value can be achieved based on either once daily administration or based on administration every 12 hours or every 8 hours. Similarly, the oral dosage capsules can be formulated such that, upon single administration to a human male, they provide a serum testosterone undecanoate C_{avg} of about 1.5 ng/mL to about 1 mcg/mL. In a further embodiment, the oral dosage capsules can be formulated such that, upon single administration to a human male, they provide a serum testosterone undecanoate C_{avg} of about 3 ng/mL to about 850 ng/mL. In a further embodiment, the oral dosage capsules can be formulated such that, upon single administration to a human male, they provide a serum testosterone undecanoate C_{avg} of about 10 ng/mL to about 850 ng/mL.

In another aspect, the oral dosage capsules can be formulated such that upon single administration d to a male human subject they provide a ratio of serum testosterone undecanoate C_{avg} to serum total testosterone C_{avg} of about 4:1 to about 75:1. In a further embodiment, the oral dosage capsules can be formulated such that, upon single administration to a human male, they provide a ratio of serum testosterone undecanoate C_{avg} to serum total testosterone C_{avg} of about 20:1 to about 50:1. In yet another embodiment, the oral dosage capsules can be formulated such that, upon single administration to a human male, the oral dosage capsule provides a ratio of serum total testosterone C_{avg} to dose of testosterone undecanoate of about 0.2×10⁻⁶ dL⁻¹ to about 20×10⁻⁶ dL⁻¹.

In one embodiment, a single dose of the testosterone undecanoate composition or oral dosage form can provide a serum total testosterone C_{avg} of about 300 ng/dL or more from about 0.5 hours to about 24 hours after oral administration with a meal. In a further embodiment, a single dose of a testosterone undecanoate composition or oral dosage capsule can provide a serum total testosterone C_{avg} of about 300 ng/dL or more at about 20 hours after oral administration with a meal. In yet a further embodiment, a single dose of the testosterone undecanoate composition can provide a serum total testosterone C_{avg} of about 300 ng/dL or more at about 18 hours after oral administration with a meal. In still a further embodiment, a single dose of the testosterone undecanoate composition can provide a serum total testosterone C_{avg} of about 300 ng/dL or more at about 16 hours after oral administration with a meal. In still a further embodiment, a single dose of the testosterone undecanoate composition can provide a serum total testosterone C_{avg} of about 300 ng/dL or more at about 12 hours after administration after oral administration with a meal. In still a further embodiment, a single dose of the testosterone undecanoate composition can provide a serum total testosterone C_{avg} of about 300 ng/dL or more at about 8 hours after oral administration with a meal. The meal that is administered with the composition or oral dosage form can be a standard meal.

The compositions and oral dosage capsules disclosed herein can be, but do not have to be, orally administered with food. In one embodiment, the composition or oral dosage capsule can be administered with a meal, such as a meal that provides about 200 to about 1000 calories of energy. In another embodiment, the composition or oral dosage capsule can be administered with a standard meal. In another embodiment, the composition or oral dosage capsule can be administered with a meal that provides about 50% of the calories derived from the fat. In another embodiment, the composition or oral dosage capsule can be administered with a high-fat, high calorie meal. In another embodiment, the composition or oral dosage capsule can be administered with a meal that provides about 500 to about 1000 calories of energy. In another embodiment, the composition or oral dosage capsule can be administered with a meal that provides about 400 to about 700 calories derived from the fat therein. The compositional make-up of the meals that are administered can vary depending on the tastes and dietary needs of a subject. However, in some situations it may be beneficial to administer the compositions and oral dosage forms with meals that provide no fat or up to about 50 g of fat. In one embodiment, the meal can provide about 10 g to about 50 g of fat. In yet a further embodiment, the meal can provide about 30 g of fat. The testosterone undecanoate dosage compositions and oral dosage capsules disclosed herein can be orally administered in a 24 hours' dosing regimen (also referred to as or a daily dosing regimen) that is suitable to the needs of the subject. The 24 hours' dosing regimen can include administering the dosage forms after meals in the morning, at about noon, in the evening, at about night time or combinations thereof. The 24 hours' dosing regimen can include dosing one or more dosage units at one or more administration times. In one embodiment, the pharmaceutical composition is administered as a single oral dosage capsule.

The testosterone undecanoate compositions and oral dosage capsules can provide increased bioavailability as compared to other testosterone undecanoate compositions and dosage forms. In some embodiments, the testosterone undecanoate oral dosage capsules can provide an in vitro release of less than about 85 wt % of the testosterone undecanoate within the first 30 minutes. The in vitro release is determined in about 1000 mL of 8% w/v Triton X-100 in water maintained at about 37° C. in an USPType-2 Apparatus at about 100 rpm. It has been discovered that these testosterone undecanoate oral dosage capsules, i.e. those having the above release characteristics, provide at least a 10% increase in the testosterone undecanoate AUC after single oral dosages are administered to human males. The increase is as compared to equivalent dosages of testosterone undecanoate in an immediate release dosage forms administered under same conditions. Immediate release dosage forms are defined as being dosage forms which release more than 85 wt % of the testosterone undecanoate within the first 30 minutes using the same in vitro release conditions described above. Further, in one embodiment, the testosterone undecanoate oral dosage capsules can provide at least a 15% increase in the testosterone undecanoate AUC as compared to an immediate release dosage oral dosage form.

In another embodiment, the testosterone undecanoate oral dosage capsules disclosed herein can provide at least a 10% reduction in the inter-subject variability of the testosterone undecanoate Cₘₐₓ, and/or the testosterone undecanoate AUC as compared to immediate release equivalent dosage containing oral dosage forms. In another embodiment, the testosterone undecanoate oral dosage capsules disclosed herein can provide 10% or more testosterone bioavailability in subjects as compared to immediate release equivalent dosed oral dosage forms.

The testosterone undecanoate compositions and oral dosage capsules disclosed herein can be used in conjunction with or as a component of a diagnostic or treatment kit that enables the diagnosis and treatment of a male patient in need of testosterone therapy. The diagnostic or treatment kit may comprise the testosterone undecanoate composition or oral dosage capsule disclosed herein along with one or more other components, including, but not limited to 1) instructions to enable those ordinarily skilled in the art to prepare a dosage form for immediate dispensing to the subject in need of; 2) one or more containers filled with one or more of the ingredients of the oral pharmaceutical dosage forms of the invention. Suitable containers include, for example, a bottle, a box, a blister card, a foil packet, or a combination thereof; 3) a tamper proof container or packaging; 4) other pharmaceutical dosage forms including other active agents including PDE-5 inhibitors and glucocorticosteroids; 5) Notice or printed instructions: in a form prescribed by a governmental agency regulating the manufacture, use, or sale of pharmaceuticals or biological products, which notice reflects approval by the agency of the manufacture, use, or sale for human administration to treat a condition that could be treated by oral testosterone therapy; 6) A “planner” for monitoring and tracking administration of the oral dosage forms; 7) Containers for storing and transporting the components of the kit; 8) total testosterone or free testosterone testing kits; 9) Sex Hormone binding globulin, SHBG, testing kits; 10) Body mass index testing materials to identify high risk patients; 11) tests for identifying patients with hypogonadism; 12) tests to assess testicular function or impotency; 13) test for bone mineral density/osteoporosis; 14) test for hair density 15) test for muscle mass and strength; 16) test for determining erectile dysfunction; 17) test for decreased libido; 18) test for fatigue, depression, mood disorders or irritability; 19) test for infertility; 20) test for prostate condition.

The oral dosage compositions and oral dosage capsules disclosed herein can be co-administered with other active agents in order to treat a target condition. One or more co-administered active agents can be admixed with the testosterone undecanoate containing compositions and/or oral dosage forms of the current invention. For example, phosphodiesterase type 5 (PDE-5) inhibitors, such as sildenafil citrate, tadalafil, vardenafil avanafil, lodenafil, mirodenafil, udenafil, and the like, are used to block the degradative action of phosphodiesterase type 5 enzyme on cyclic GMP in the smooth muscle cells lining the blood vessels supplying the corpus cavernosum of the penis and are frequently used to treat erectile dysfunction. Such compounds could be co-administered with the compositions and oral dosage forms of the present invention in order to provide improved clinical outcomes through synergistic pharmacological action as measured by improved (sooner, better and longer lasting) erection, potency, libido, mood, body mass, etc. in males relative to administration of the testosterone or the co-administered PDE-5 alone. The testosterone undecanoate compositions and oral dosage capsules can also be co-administered with one or more other active agents such as aromatase inhibitors (for example letrozole, anastrozole, exemestane, fadrozole, vorozole, formestane etc.), dopamine agonists (for example apomorphine, bromocriptine, cabergoline, pergolide, ropinirole, rotigotine, pramipexole, fenoldopam etc.), prostaglandins (for example alprostadil), alpha blockers (for example yohimbine, phentolamine), vasodilators (for example minoxidil) and the like, for improved clinical outcomes through synergistic pharmacological action as measured by improvements in one or more of the secondary sexual characteristics in males such as sexual activity, potency, libido, erection etc., mood, body mass and the like, relative to administration of either the testosterone or the co-administered active agent alone.

In a further aspect, the compositions of the current invention can be formulated to provide a gastro-retentive dosage form. In one embodiment, the gastro-retentive dosage form is a capsule. In another embodiment, the gastro-retentive dosage form is retained in the upper gastro-intestinal tract for at least one hour post-dosing. In another embodiment, the gastro-retentive dosage form is retained in the upper gastro-intestinal tract for at least two hours post-dosing. In another embodiment, the gastro-retentive dosage form is retained in the upper gastro-intestinal tract for at least 4 hours post-dosing. In another embodiment, the gastro-retentive dosage form is formulated to float in the stomach after dosing. In another embodiment, the gastro-retentive dosage form is formulated to expand when it comes in contact with aqueous medium to at least 1.3 times its size compared to its size when it is not in contact with the aqueous use environment. In another embodiment, the gastro-retentive dosage form is formulated to adhere to the lining of the stomach wall after dosing.

The compositions and the oral dosage capsules of the current invention can also include one or more of other additives selected from binders, bufferants, diluents, disintegrants, flavors, colorants, taste-masking agents, resins, pH modifiers, lubricants, glidants, thickening agent, opacifying agent, humectants, desiccants, effervescing agents, plasticizing agents and the like.

### EXAMPLES

The following examples are provided to promote a more clear understanding of certain embodiments of the present invention, and are in no way meant as a limitation thereon.

### Example 1

### Testosterone Undecanoate Composition

A testosterone undecanoate containing composition was prepared by using the components set forth in Table I. The composition is prepared by weighing all of the components, except the testosterone undecanoate, into a clean stainless steel container and mixed together at about 50° C. to about 70° C., using a stirrer. The testosterone undecanoate (TU) is added and stirred into the mixture of other components until the testosterone undecanoate dissolves. A predetermined quantity of this “liquid fill material” is disposed into a capsule (for example, hard gelatin capsule) to get the required testosterone undecanoate dose per dosage unit. The capsules are allowed to cool at room temperature, banded (if required) and packaged in a HDPE bottle and tightly closed with an appropriate lid.

TABLE I

| Example 1 | Composition mg/capsule |
|---|---|
| Testosterone Undecanoate | 200 |
| Solubilizer (e.g. Glycerides of | 725 |
| coconut oil; Capmul ® MCM) | |
| Dispersant (e.g. lauroglycol) | 300 |
| Dispersant (polyoxyl 40 hydrogenated | 50* |
| castor oil or Cremophor ® RH40) | |

Drug Loading per capsule = 15.7%
*contributes to non-appreciable TU solubilization in the composition

### Example 2

### Testosterone Undecanoate Composition

A testosterone undecanoate containing composition was prepared similarly to the method described in Example 1 using the components set forth in Table II.

TABLE II

| Example 2 | Composition mg/capsule |
|---|---|
| Testosterone Undecanoate | 225 |
| Solubilizer (e.g. Maize oil glyceride) | 260 |
| Dispersant (e.g. lauroglycol) | 665 |

Drug Loading per capsule~19.6%

### Examples 3 & 4

### Testosterone Undecanoate Composition

Testosterone undecanoate containing composition were prepared similarly to the method described in Example 1 using the components set forth in Tables III and IV

TABLE III

| Example 3 | Composition mg/capsule |
|---|---|
| Testosterone Undecanoate | 200 |
| Solubilizer (e.g. Glycerides of | 600 |
| coconut oil; Capmul ® MCM) | |

Drug Loading per capsule = 25%

TABLE IV

| Example 4 | Composition mg/capsule |
|---|---|
| Testosterone Undecanoate | 180 |
| Solubilizer (Maize oil glyceride, | 600 |
| Maisine 35-1) | |

Drug Loading per capsule = 23%

### Example 5

### Testosterone Undecanoate Composition

A testosterone undecanoate containing composition was prepared similarly to the method described in Example 1 using the components set forth in Table V

TABLE V

| Example 5 | Composition mg/capsule |
|---|---|
| Testosterone Undecanoate | 240 |
| Solubilizer (e.g. Glycerides of | 200 |
| coconut oil; Capmul ® MCM) | |
| Solubilizer (e.g. α-tocopherol) | 490 |
| Dispersant (for e.g. polyoxyl castor | 100* |
| oil or Cremophor ® EL) | |

TU- Loading per capsule = 23.3%
*contributes to non-appreciable TU solubilization in the composition

### Example 6

### Testosterone Undecanoate Composition

A testosterone undecanoate containing composition was prepared by using the components set forth in Table VI and a method similar to that described in Example 1.

TABLE VI

| Example 6 | Composition mg/capsule |
|---|---|
| Testosterone Undecanoate | 200 |
| Solubilizer (e.g. Maize oil glycerides) | 490 |
| Dispersant (e.g. polysorbate 80) | 25* |
| Solidifying agent (e.g. polyethylene | 45 |
| glycol, 8000 or PEG 8000) | |

TU- Loading per capsule = 26.3%
*contributes to non-appreciable TU solubilization in the composition;

### Example 7

### Testosterone Undecanoate Composition

A testosterone undecanoate containing composition was prepared by using the components set forth in Table VII and a method similar to that described in Example 1.

TABLE VII

| Example 7 | Composition mg/capsule |
|---|---|
| Testosterone Undecanoate | 240 |
| Solubilizer (e.g. Maize oil glycerides) | 325 |
| Solubilizer (e.g. oleic acid) | 125 |
| Solubilizer (e.g. Benzyl Alcohol) | 50 |
| Solubilizer (e.g. α-tocopherol) | 75 |
| Solidifying agent (e.g. PEG 8000) | 45 |

TU- Loading per capsule = 28%

### Example 8

### Testosterone Undecanoate Composition

A testosterone undecanoate containing composition was prepared by using the components set forth in Table VIII and a method similar to that described in Example 1.

TABLE VIII

| Example 6 | Composition mg/capsule |
|---|---|
| Testosterone Undecanoate | 240 |
| Solubilizer (e.g. oleic acid) | 400 |
| Solidifying agent- (e.g. PEG 8000) | 45 |

TU- Loading per capsule = 35%

### Example 9

### Testosterone Undecanoate Composition

A testosterone undecanoate containing composition was prepared using the components set forth in Table IX and a method similar to that described in Example 1.

TABLE IX

| Example 7 | Composition mg/capsule |
|---|---|
| Testosterone Undecanoate | 240 |
| Solubilizer (e.g. Maize oil glycerides) | 400 |
| Solubilizer (e.g. α-tocopherol) | 24 |
| Solidifying agent -(e.g. Glyceryl | 25 |
| distearate; Percirol ® ATO 5) | |

TU- Loading per capsule = 34.8%

### Example 10

### Testosterone Undecanoate Composition

A testosterone undecanoate containing composition can be prepared by using the components set forth in Table X by a method as follows: The required quantity of the glyceryl distearate or glyceryl monostearate and the PEG 8000 are placed in a stainless steel container and heated to about 50 to 70° C. to get a molten mixture. The testosterone undecanoate is added and stirred till it completely dissolves. A predetermined weight of the molten mixture is disposed into capsules and allowed to congeal at room temperature, banded and packed.

TABLE X

| Example 10 | Composition mg/capsule |
|---|---|
| Testosterone Undecanoate | 100 |
| Glyceryl distearate (Percirol ® | 200 |
| ATO 5) or glyceryl monostearate | |
| PEG 8000 | 50 |

The oral dosage capsules of Example 10 can provide, upon single administration along with food to a human male, a testosterone undecanoate AUC that is about 20% higher as compared to a composition that does not include the glyceryl distearate (Percirol® ATO 5) or glyceryl monostearate.

The composition of Example 10 can also be optionally modified so that a dispersant such as a disintegrating agent (e.g. Crospovidone at about 150 mg for every 100 mg TU dose) can be uniformly suspended under stirring in the molten testosterone undecanoate solution. This suspension can be further allowed to cooled and passed through ASTM 30 mesh get granulates or particulates which can be either filled in a capsule or compressed to a tablet.

### Example 11

### Testosterone Undecanoate Composition

A testosterone undecanoate containing composition is prepared by using the components set forth in Table XI and a method similar to that described under Example 1.

TABLE XI

| Example 11 | Composition mg/capsule |
|---|---|
| Testosterone Undecanoate | 225 |
| Solubilizer (e.g. Castor Oil) | 350 |
| Dispersant (e.g. lauroglycol); | 180 |
| Solidifying agent (e.g. PEG 8000) | 45 |

TU- Loading per capsule = 28.1%

### Examples 12-19

### Testosterone Undecanoate Compositions

Testosterone undecanoate formulations of Examples 12 through 19 were prepared by using the components set forth in Table XII and by a method similar to that described under Example 1. Additionally, indicated amounts of the respective formulations were filled into hard gelatin capsules and the testosterone undecanoate release from capsules is measured using a USP Type-II apparatus at about 100 rpm in about 1000 mL of 8% w/w solution of Triton X100 in water, maintained at about 37° C. The results of the release testing are also shown in Table XII.

TABLE XII

| | Composition mg/capsule | | | | | | | |
|---|---|---|---|---|---|---|---|---|
| Capsule Components/ | Example | Example | Example | Example | Example | Example | Example | Example- |
| Attributes | 12 | 13 | 14 | 15 | 16 | 17 | 18 | 19 |
| Testosterone | 40 | 40 | 75 | 75 | 75 | 75 | 75 | 125 |
| Undecanoate | | | | | | | | |
| Oleic Acid | 227 | — | — | — | — | — | — | |
| Castor oil | | 175 | — | — | 455 | — | — | |
| Lauroglycol | | 115 | — | — | — | — | — | |
| Labrafil M2125CS | | | | | 80 | — | — | |
| Maize oil glycerides | | — | 455 | 455 | — | 316 | 316 | 515 |
| (Maisine 35-1) | | | | | | | | |
| Polyoxyl 40 | | — | 130 | 80 | — | 79 | 54 | 112 |
| Hydrogenated Castor | | | | | | | | |
| Oil, (Cremophor | | | | | | | | |
| RH40) | | | | | | | | |
| Glyceryl distearate | | — | — | 50 | — | — | 25 | |
| (Percirol ATO 5) | | | | | | | | |
| Polyethylene Glycol | | — | — | — | 48 | 30 | 30 | 48 |
| 8000, | | | | | | | | |
| Total mg per unit | 267 | 330 | 660 | 660 | 660 | 500 | 500 | 800 |
| capsule (mg) | | | | | | | | |
| TU- solubilized per | 15% | 12% | 11.3% | 11.3% | 11.3% | 15% | 15% | 15.6% |
| unit (%) | | | | | | | | |
| TU Fully Solubilized | Yes | Yes | Yes | Yes | Yes | Yes | Yes | Yes |
| TU released in 30 | ~100% | ~100% | ~100% | 30% | ~78% | 85% | 32% | 80% |
| minutes (%) | | | | | | | | |
| Time for 75% TU | <120 | <120 | <120 | <120 | <120 | <120 | <120 | <120 |
| release (minutes) | | | | | | | | |

### Examples 20-25

### Testosterone Undecanoate Compositions

Testosterone undecanoate formulations of Examples 20 through 25 were prepared by using the components set forth in Table XIII and by a method similar to that described under Example 1. Additionally, indicated amounts of the respective formulations were filled into hard gelatin capsules and the testosterone undecanoate release from capsules was tested in about 1000 mL of 8% w/w solution of Triton X100 in water, maintained at about 37° C., using a USP Type-II apparatus at about 100 rpm. The results of the release testing are also shown in Table XIII.

TABLE XIII

| | Composition mg/capsule | | | | | |
|---|---|---|---|---|---|---|
| Capsule Components/ | Example | Example | Example | Example | Example | Example |
| Attributes | 20 | 21 | 22 | 23 | 24 | 25 |
| Testosterone | 200 | 200 | 240 | 240 | 240 | 240 |
| Undecanoate (TU) | | | | | | |
| Maize oil glycerides | — | 490 | 464 | 464 | | 304 |
| (Maisine 35-1) | | | | | | |
| Coconut oil glycerides | — | — | — | — | 400 | — |
| (Capmul MCM) | | | | | | |
| Alpha- tocopherol | 510 | — | — | 50 | — | 50 |
| Benzyl alcohol | — | | — | — | 25 | 50 |
| Polyoxyl 40 | — | 25 | — | — | — | — |
| Hydrogenated Castor | | | | | | |
| Oil, | | | | | | |
| Polyoxyl 35 Castor Oil, | 45 | — | — | — | — | — |
| Polyethylene Glycol | 45 | 45 | — | 46 | 65 | 41 |
| 8000, USP | | | | | | |
| Total Fill wt. per unit | 800 | 760 | 704 | 800 | 730 | 685 |
| (mg) | | | | | | |
| % TU- loading per unit | 25 | 26.3 | 34.0 | 30.0 | 32.9 | 35.0 |
| TU Fully solubilized | Yes | Yes | Yes | Yes | Yes | Yes |
| TU released in 30 | 43% | 62% | 38% | 32% | <75% | <75% |
| minutes | | | | | | |
| Time for 75% TU | <120 | <120 | <120 | <120 | <120 | <120 |
| release (minutes) | | | | | | |

### Examples 26-29

Testosterone undecanoate formulations of Examples 26 through 29 were prepared by using the components set forth in Table XIV and a method similar to that described under Example 1. Additionally, indicated amounts of the respective formulations were filled into hard gelatin capsules and the testosterone undecanoate released from capsules was tested in about 1000 mL of 8% w/w solution of Triton X100 in water, maintained at about 37° C., using a USP Type-II apparatus at about 100 rpm. The calculated results of the release testing are also shown in Table XIV.

TABLE XIV

| | Composition mg/capsule | | | |
|---|---|---|---|---|
| Capsule Components/ | Example | Example | Example | Example |
| Attributes | 26 | 27 | 28 | 29 |
| Testosterone | 250 | 250 | 250 | 250 |
| Undecanoate | | | | |
| Maize oil glycerides | 486 | 937 | 410 | 939 |
| (Maisine 35-1) | | | | |
| Polyoxyl 40 | 25 | 213 | 69 | 144 |
| Hydrogenated Castor | | | | |
| Oil (Cremophor RH40) | | | | |
| Glyceryl distearate | — | — | 32 | 67 |
| (Percirol ATO 5) | | | | |
| Polyethylene Glycol | 39 | — | 39 | — |
| 8000, | | | | |
| Total mg per unit capsule | 800 | 1400 | 800 | 1400 |
| % TU loading | 31.3 | 18 | 31.3 | 18 |
| TU released in 30 | <75% | <75% | <75% | <75% |
| minutes | | | | |
| Time for 75% TU | <120 | <120 | <120 | <120 |
| release (minutes) | | | | |

Note:
Examples 27 and 29 can optionally be disposed in a delayed release capsule

### Examples 30-35

Testosterone undecanoate formulations Examples 30 through 35 can be prepared by using the components set forth in Table XV and by a method similar to that described in Example 1. Additionally, indicated amounts of the respective formulations can be encapsulated in gelatin capsules and the testosterone undecanoate release from the capsules tested in about 1000 mL of 8% w/w solution of Triton X100 in water, maintained at about 37° C., using a USP Type-II apparatus at about 100 rpm. The calculated results of the release testing are also shown in Table XV.

TABLE XV

| | Composition mg/capsule | | | | | |
|---|---|---|---|---|---|---|
| Capsule Components/ | Example | Example | Example | Example | Example | Example |
| Attributes | 30 | 31 | 32 | 33 | 34 | 35 |
| Testosterone | 368 | 320 | 490 | 240 | 40 | 300 |
| Undecanoate | | | | | | |
| Maize oil glycerides | 900 | 370 | 620 | 404 | — | — |
| (Maisine 35-1) | | | | | | |
| Castor Oil | — | — | — | — | 175 | 276 |
| Lauroglycol | — | — | | — | 115 | 184 |
| Tocopherol | | | 102 | — | — | |
| Benzyl alcohol | — | — | 102 | — | — | |
| Polyoxyl 40 | 46 | 25 | — | — | — | — |
| Hydrogenated Castor | | | | | | |
| Oil, | | | | | | |
| Polyethylene Glycol | 86 | 45 | 86 | 41 | — | — |
| 8000, USP | | | | | | |
| % TU- per unit fill | 26.3 | 36.3 | 35.0 | 35.0 | 12.0 | 39.5 |
| Total Fill wt. per unit | 1400 | 760 | 1400 | 685 | 330 | 760 |
| TU Fully solubilized | Yes | NO | Yes | No | Yes | No |
| TU released in 30 | 62% | <62% | <75% | <75% | ~100% | <75% |
| minutes | | | | | | |
| Time for 75% TU | <120 | >120 | <120 | <120 | <120 | >120 |
| release (minutes) | | | | | | |

Examples 30 through 35 demonstrate the importance of the choice of the solubilizers of the current invention and their levels to achieve greater testosterone undecanoate loading and yet maintain the solubilization of the testosterone undecanoate in the composition and/or the dosage form.

### Examples 36 and 37

### Testosterone Undecanoate Containing Compositions

The compositions of the current invention can be further adsorbed onto one or more substrate materials such as, for example, lactose, magnesium aluminosilicate, colloidal silicon dioxide, starch, microcrystalline cellulose, alkyl celluloses etc., whereby a free flowing powder/granule form is obtained which can be used as a granules, or disposed into capsule, or pressed into tablet. The amount of the substrate material can be from about 15% to about 40% of the weight of the composition. In one embodiment, the amount of the substrate material can be from about 20% to about 35% of the weight of the formed granule or powder. The method of making such adsorbed testosterone undecanoate compositions can include pouring the liquid compositions on the substrate material under and continuous mixing at room temperature or at about 50° C.-70° C., depending on the composition. After cooling, the adsorbed composition can be disposed into capsule or pressed into tablet. Table XVI illustrates examples of the freely flowable adsorbed solubilized Testosterone Undecanoate compositions.

TABLE XVI

| | Composition (% w/w) | |
|---|---|---|
| Components/Attributes | Example 36 | Example 37 |
| Testosterone Undecanoate | 16 | 16 |
| Maize oil glycerides (Maisine 35-1) | 50 | — |
| Castor Oil | — | 45 |
| Sorbitan monolaurate (Span ® 20) | — | 5 |
| Tocopherol | 1 | — |
| Glycerylpalmito stearate | 5 | — |
| Polyoxyl 40 Hydrogenated Castor Oil, | 3 | — |
| Polyethylene Glycol 8000, USP | — | 4 |
| Magnesium aluminosilicate | 25 | 30 |
| (Neusilin ® US2) | | |

### Example 38

### Stability of Testosterone Undecanoate Containing Compositions

A preliminary stability evaluation with respect to the change in potency and/or appearance of the potential primary degradation products was carried out with the compositions of Example 17 and Example 21, both filled in hard gelatin capsules at 200 mg/per capsule and 75 mg per capsules. The capsules were packed in HDPE bottles and staged for stability studies in the environmental chambers maintained at 25° C./60% RH. The primary degradation products were determined by a HPLC analysis method after about three months' storage and the results shown in Table XVII.

TABLE XVII

| TU composition | Degradant |
|---|---|
| Example- 17 | 0.15% |
| Example- 21 | 0.06% |

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

1. A pharmaceutical composition, comprising:
testosterone undecanoate, and
a solubilizer,
wherein the testosterone undecanoate is solubilized in the composition and comprises about 14 wt % to about 35 wt % of the total composition.

2. The pharmaceutical composition of claim 1, wherein the solubilizer is selected from the group consisting of C₈ to C₂₂ fatty acid glycerides, alcohols, C₈ to C₂₂ fatty acids, omega fatty acids, and mixtures thereof.

3. The pharmaceutical composition of claim 1, wherein the solubilizer comprises about 50 wt % to about 86 wt % of the composition.

4. The pharmaceutical composition of claim 1, wherein the solubilizer comprises about 60 wt % to about 80 wt % of the composition.

5. The pharmaceutical composition of claim 2, wherein the C₈ to C₂₂ fatty acid glycerides include at least about 40 wt % of monoglycerides.

6. The pharmaceutical composition of claim 2, wherein the C₈-C₂₂ fatty acid glycerides are selected from the group consisting of monoglycerides and diglycerides of capric acid, monoglycerides and diglycerides of caprylic acid, and combinations thereof.

7. The pharmaceutical composition of claim 2, wherein the C₈ to C₂₂ fatty acid glycerides are selected from the group consisting of monoglycerides and diglycerides of linoleic acid, monoglycerides and diglycerides of oleic acid, and combinations thereof.

8. The pharmaceutical composition of claim 2, wherein the C₈ to C₂₂ fatty acid glycerides are derived from sources selected from the group consisting of maize oil, poppy seed oil, safflower oil, sunflower oil, coconut oil, palm kernel oil, castor oil, and mixtures thereof.

9. The pharmaceutical composition of claim 1, wherein the solubilizer includes a C₈ to C₂₂ fatty acid.

10. The pharmaceutical composition of claim 9, wherein the C₈ to C₂₂ fatty acid is selected from the group consisting of capric acid, pelargonic acid, caprylic acid, undecanoic acid, lauric acid, myristic acid, palmitic acid, stearic acid, oleic acid, linoleic acid, linolenic acid arachodonic acid, eicosapentaenoic acid, docosahexanoic acid, and mixtures thereof.

11. The pharmaceutical composition of claim 9, wherein the C₈ to C₂₂ fatty acid is selected from the group consisting of capric acid, caprylic acid, linoleic acid, oleic acid, and mixtures thereof.

12. The pharmaceutical composition of claim 1 wherein the testosterone undecanoate has a solubility in the solubilizer at about 37° C. of about 250 mg/g to about 750 mg/g.

13. The pharmaceutical composition of claim 1, wherein the solubilizer includes a medium and/or long chain diglyceride

14. The pharmaceutical composition of claim 1, wherein the composition includes less than 45 wt % castor oil.

15. The pharmaceutical composition of claim 1, wherein the testosterone undecanoate comprises about 16 wt % to about 30 wt % of the composition.

16. The pharmaceutical composition of claim 1, wherein the testosterone undecanoate comprises about 18 wt % to about 25 wt % of the composition.

17. The pharmaceutical composition of claim 1, wherein the composition includes a dispersant.

18. The pharmaceutical composition of claim 17, wherein the dispersant is a lipophilic surfactant.

19. The pharmaceutical composition of claim 18, wherein the lipophilic surfactant is selected from the group consisting of propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate, sorbitan monopalmitate, sorbitan monooleate, sorbitan monostearate, sorbitan tristearate, sorbitan monolaurate, sorbitan monopalmitate, sorbitan monooleate, sorbitan trioleate, and combinations thereof.

20. The pharmaceutical composition of claim 17, wherein the dispersant is a hydrophilic surfactant

21. The pharmaceutical composition of claim 20, wherein the hydrophilic surfactant has at least one characteristic of
1) the hydrophilic surfactant is present in an amount such that it does not appreciably solubilize the testosterone undecanoate present in the composition, or
2) the solubility of testosterone undecanoate in the hydrophilic surfactant at about 25° C., 50 mg/gram or less, based on the total weight of the testosterone undecanoate and the solubilizer.

22. The pharmaceutical composition of claim 1, wherein the solubilizer is an alcohol.

23. The pharmaceutical composition of claim 22, wherein the alcohol is selected from the group consisting of tocopherol, benzyl alcohol, ethyl alcohol, isopropanol, butanol, ethylene glycol, propylene glycol, butanediol, glycerol, pentaerythritol, transcutol, dimethyl isosorbide, polyethylene glycol, and mixtures thereof.

24. The pharmaceutical composition of claim 22, wherein the solubilizer is ethyl alcohol benzyl alcohol, tocopherol, and mixtures thereof.

25. The pharmaceutical composition of claim 1, wherein the composition is a solid at about 20° C.

26. The pharmaceutical composition of claim 1, wherein the composition further includes a solidifying agent.

27. The pharmaceutical composition of claim 26, wherein the solidifying agent is selected from the group consisting of polyethylene glycols; sorbitol; gelatin; stearic acid; cetyl alcohol; cetosterayl alcohol; paraffin wax; polyvinyl alcohol; glyceryl stearates; glyceryl distearate; glyceryl monostearate; glyceryl palmitostearate; glyceryl behenate; waxes; hydrogenated castor oil; hydrogenated vegetable oil; bees wax, microcrystalline wax; sterols; phytosterols; cholesterol and mixtures thereof.

28. The pharmaceutical composition of claim 26, wherein the solidifying agent comprises about 0.1 wt % to about 25 wt % of the composition.

29. The pharmaceutical composition of claim 26, wherein the solidifying agent comprises about 3 wt % to about 20 wt %.

30. The pharmaceutical composition of claim 26, wherein the solidifying agent comprises about 6 wt % to about 15 wt % of the composition.

31. The pharmaceutical composition of claim 1, wherein when stored for a period of 3 months there is at least 20% less degradation of the testosterone undecanoate in the composition as compared to compositions comprising less than 14 wt % testosterone undecanoate.

32. The pharmaceutical composition of claim 15, wherein when stored for a period of 3 months there is at least 20% less degradation of the testosterone undecanoate in the composition as compared to compositions comprising less than 16 wt % testosterone undecanoate.

33. A pharmaceutical oral dosage capsule, comprising:
the pharmaceutical composition of claim 1 disposed in a capsule shell.

34. The oral dosage capsule of claim 33, wherein the ratio of the amount of testosterone undecanoate in the dosage form to the volume of the capsule fill is about 80 mg/mL to about 750 mg/mL.

35. The oral dosage capsule of claim 33, wherein the ratio of the amount of testosterone undecanoate to the volume of the capsule fill is about 160 mg/mL to about 375 mg/mL.

36. The oral dosage capsule of claim 33, wherein the testosterone undecanoate is present in an amount of about 80 mg to about 400 mg.

37. The oral dosage capsule of claim 33, wherein the testosterone undecanoate is present in an amount of about 120 mg to about 300 mg.

38. The oral dosage capsule of claim 33, wherein the testosterone undecanoate is present in amount of about 150 mg to about 250 mg.

39. The oral dosage capsule of claim 33, wherein the oral dosage capsule is delayed release.

40. A pharmaceutical oral dosage capsule, comprising:
at least 50 mg of testosterone undecanoate, and
a solubilizer
wherein the oral dosage capsule has a ratio of testosterone undecanoate to the volume of the capsule fill of 80 mg/mL to about 750 mg/mL, and wherein the capsule provides in vitro release of at least about 75 wt % of the testosterone undecanoate during the first 120 minutes when tested using about 1000 mL of 8% w/v Triton X-100 in water maintained at about 37±1° C. taken in a USP-Type II dissolution apparatus set at 100 rpm.

41. The oral dosage capsule of claim 40, wherein the oral dosage capsule includes 80 mg to about 400 mg of testosterone undecanoate.

42. The oral dosage capsule of claim 40, wherein the oral dosage capsule upon single administration to a human male, provides a serum total testosterone C_{avg} ranging about 300 ng/dL to about 1100 ng/dL.

43. The oral dosage capsule of claim 40, wherein the oral dosage capsule upon single administration to a human male, provides a serum total testosterone C_{avg} ranging about 350 ng/dL to about 800 ng/dL.

44. The oral dosage capsule of claim 40, wherein the oral dosage capsule upon single administration to a human male, provides a serum total testosterone C_{avg} ranging about 400 ng/dL to about 600 ng/dL.

45. The oral dosage capsule of claim 40, wherein the oral dosage capsule upon single administration to a human male, provides a serum testosterone undecanoate C_{avg} of 1.5 ng/mL to about 1 mcg/mL.

46. The oral dosage capsule of claim 40, wherein the oral dosage capsule upon single administration to a human male, provides a serum testosterone undecanoate C_{avg} of 10 ng/mL to about 850 ng/mL.

47. The oral dosage capsule of claim 40, wherein the oral dosage capsule upon single administration to a human male, provides a ratio of serum testosterone undecanoate C_{avg} to serum total testosterone C_{avg} of about 4:1 to about 75:1.

48. The oral dosage capsule of claim 40, wherein the oral dosage capsule upon single administration to a human male, provides a ratio of serum testosterone undecanoate C_{avg} to serum total testosterone C_{avg} of about 20:1 to about 50:1

49. The oral dosage capsule of claim 40, wherein the oral dosage capsule upon single administration to a human male, provides a ratio of serum total testosterone C_{avg} to dose of testosterone undecanoate of about 0.2×10⁻⁶ dL⁻¹ to about 20×10⁻⁶ dL⁻¹.

50. The oral dosage capsule of claim 40, wherein the capsule has an in vitro release profile such that 85 wt % or less of the testosterone undecanoate is released in the first 30 minutes, when measured using about 1000 mL of 8% w/v Triton X-100 in water maintained at about 37±1° C. taken in a USP-Type II dissolution apparatus set at 100 rpm.

51. The oral dosage capsule of claim 40, wherein the capsule has an in vitro release profile such that 70 wt % or less of the testosterone undecanoate is released in the first 30 minutes, when measured using about 1000 mL of 8% w/v Triton X-100 in water maintained at about 37±1° C. taken in a USP-Type II dissolution apparatus set at 100 rpm.

52. The oral dosage capsule of claim 40, wherein the capsule has a in vitro release profile such that at least 35 wt % of the testosterone undecanoate is released in the first 30 minutes, when measured using about 1000 mL of 8% w/v Triton X-100 in water maintained at about 37±1° C. taken in a USP-Type II dissolution apparatus set at 100 rpm.

53. The oral dosage capsule of claim 40, wherein the solubilizer is selected from the group consisting of C₈ to C₂₂ fatty acid glycerides, alcohols, C₈ to C₂₂ fatty acids, omega fatty acids, and mixtures thereof.

54. The oral dosage capsule of claim 53, wherein the solubilizer comprises about 50 wt % to about 86 wt % of the composition.

55. The oral dosage capsule of claim 53, wherein the solubilizer comprises about 60 wt % to about 80 wt % of the composition.

56. The oral dosage capsule of claim 53, wherein the C₈ to C₂₂ fatty acid glycerides include at least about 40 wt % of monoglycerides.

57. The oral dosage capsule of claim 53, wherein the C₈-C₂₂ fatty acid glycerides are selected from the group consisting of monoglycerides and diglycerides of capric acid, monoglycerides and diglycerides of caprylic acid, and combinations thereof.

58. The oral dosage capsule of claim 53, wherein the C₈ to C₂₂ fatty acid glycerides are selected from the group consisting of monoglycerides and diglycerides of linoleic acid, monoglycerides and diglycerides of oleic acid, and combinations thereof.

59. The oral dosage capsule of claim 53, wherein the C₈ to C₂₂ fatty acid glycerides are derived from sources selected from the group consisting of maize oil, poppy seed oil, safflower oil, sunflower oil, coconut oil, palm kernel oil, castor oil, and mixtures thereof.

60. The oral dosage capsule of claim 53, wherein when stored for a period of 3 months, there is at least 20% less degradation of the testosterone undecanoate in the oral dosage capsule as compared to an oral dosage composition comprising less than 16 wt % testosterone undecanoate.

61. A method of treating in a human male suffering from testosterone deficiency, comprising:
orally administering the pharmaceutical composition of claim 1 to a human male in need thereof.

62. The method of claim 61, wherein the administration is done every 24 hours.

63. The method of claim 61, wherein the administration is done every 12 hours.

64. The method of claim 61, wherein the pharmaceutical composition is administered as a single oral dosage capsule.

65. A method of treating in a human male suffering from testosterone deficiency, comprising:
orally administering the oral dosage capsule of claim 48 to a human male in need thereof.
